(19) United States
(12) Patent Application Publication
(10) Pub. No.: US 2026/0034110 A1
(43) Pub. Date: Feb. 5, 2026

(54) LUMACAFTOR FOR TREATING THROMBOSIS AND FOR RESTORING THE AUTOINHIBITORY FUNCTION OF VON WILLEBRAND FACTOR UNDER PRO-THROMBOTIC INFLAMMATORY CONDITIONS WHILE MAINTAINING HEMOSTASIS

(71) Applicant: University of Washington, Seattle, WA (US)

(72) Inventor: Gianluca Interlandi, Seattle, WA (US)

(73) Assignee: University of Washington, Seattle, WA (US)

(21) Appl. No.: 19/284,618

(22) Filed: Jul. 29, 2025

Related U.S. Application Data
(60) Provisional application No. 63/678,300, filed on Aug. 1, 2024.

(51) Int. Cl. A61K 31/443

(52) U.S. Cl. CPC A61K 31/443

(57) ABSTRACT

Methods for using lumacaftor or a pharmaceutically acceptable salt thereof for treating thrombosis and for restoring the autoinhibitory function of von Willebrand factor (VWF) under pro-thrombotic inflammatory conditions while maintaining hemostasis.

### CROSS-REFERENCE TO RELATED APPLICATION

This application claims the benefit of U.S. Patent Application No. 63/678,300, filed Aug. 1, 2024, expressly incorporated herein by reference in its entirety.

### BACKGROUND

Currently available anti-thrombotic therapeutic drugs, often referred to as “blood thinners”, generally reduce the ability of blood to clot irrespective of whether clotting is due to pro-thrombotic conditions leading to pathological thrombus formation or to the physiological response to vessel rupture. In fact, commonly available anti-thrombotic drugs such as warfarin, caplacizumab, which targets VWF directly and is used to treat thrombotic thrombocytopenia purpura, or apixaban (sold as Eliquis®), an inhibitor of factor Xa, all carry the risk of bleeding as side effects. Hence the question: is it possible to design a therapeutic that blocks the formation of a pathological thrombus without affecting the beneficial haemostatic response of blood to clot in case of injury? Such a drug would need to distinguish between an inflammatory pro-thrombotic environment and a situation where clotting is activated to stop blood loss.

Inflammation has been linked to an increased risk of thrombosis. During the inflammatory response, hydrogen peroxide is released, which is converted to hypochlorous acid (HOCI) through the action of myeloperoxidase. The oxidizing agent HOCI causes the conversion of methionine residues to methionine sulfoxide modifying the structure and function of several blood proteins inducing a pro-thrombotic state. An example of a blood protein whose pro-thrombotic function has been shown to be increased following oxidation of its methionine residues is von Willebrand factor (VWF). Multiple studies have investigated VWF as a possible target of anti-thrombotic therapies because VWF is thought to have a central role in pathological thrombus formation. Hence, studying how VWF is activated under various conditions in particular in the presence of oxidants may provide a pathway how to design an inhibitor that targets VWF selectively under a pro-thrombotic inflammatory environment.

The protein VWF tethers blood platelets to the site of vascular injury in the early stages of hemostasis. It can be described as a relatively long multimeric chain where monomers are linked to each other through disulfide bonds at the N- and C-terminii with each monomer consisting of a number of domains that are covalently linked to each other (See FIG. 1). The A1 domain is responsible for mediating the platelet-binding function of VWF by binding to glycoprotein Ibα (GpIbα) located on the surface of blood platelets. It has been shown that the function of the A1 domain can be inhibited by its neighboring domains, specifically, the D′D3 domains located N-terminally and the A2 and A3 domains located C-terminally. Normally, tensile force generated by shear stress present in flowing blood separates neighboring domains from A1, which becomes exposed and is able to bind GpIbα. Recently, oxidizing conditions have been shown to increase the platelet-binding function of VWF and this has been linked to the oxidation of methionine residues in the A1, A2 and A3 domains. Furthermore, molecular dynamics (MD) studies have provided evidence that oxidation of methionine residues destabilizes the fold of the A2 domain and disrupts the interface between the A1 and A2 domains. The separation of the A1 and A2 domains from each other due to methionine oxidation is consistent with experimental evidence that oxidation increases the GpIbα-binding function of a A1A2A3 domain construct but not of isolated A1. Hence, studying how methionine oxidation alters the interfaces between A1 and the neighboring domains, which inhibit its function, may lead to the discovery of therapeutics that block VWF function only under inflammation-induced oxidizing conditions while preserving the hemostatic function of VWF.

A need exists to develop tools to efficiently screen for drugs that compensate the activating effect of oxidation on VWF while leaving the platelet-binding function of unoxidized VWF unaltered. This would be useful for example to search for drugs that are already available or even already approved by the Food and Drug Administration (FDA) and could be repurposed as anti-thrombotic therapeutics if they have the desired effect on VWF.

There is also a need for therapeutics that are more effective in inhibiting VWF under oxidizing than under normal conditions.

The present disclosure seeks to fulfill these needs and provides further related advantages.

### SUMMARY

In one aspect, the present disclosure provides lumacaftor as a VWF ligand that prevents thrombosis while maintaining hemostasis. In the method, a therapeutically effective amount of lumacaftor or a pharmaceutically acceptable salt thereof is administered to a subject in need thereof.

In another aspect, the disclosure provides a method for restoring the autoinhibitory function of von Willebrand factor (VWF) under pro-thrombotic inflammatory conditions. In the method, lumacaftor or a pharmaceutically acceptable thereof binds to a methionine sulfoxide at the A1-A2 inter-domain interface of VWF. The method comprises contacting VWF with an amount of lumacaftor or a pharmaceutically acceptable thereof effective to bind to a methionine sulfoxide at the A1-A2 inter-domain interface of VWF. In certain embodiments, the methionine sulfoxide to which lumacaftor or a pharmaceutically acceptable thereof binds is the oxidized form of M1495.

In a further aspect, the present disclosure provides a method based on an enzyme-linked immunosorbent assay (ELISA) to measure the binding activity of VWF to GpIbα comparing oxidized and unoxidized VWF in the absence and the presence of a therapeutic candidate.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic representation of a VWF monomer indicating substrates of the A1 (GpIbα) and A3 (collagen) domains and illustrating that the A2 domain contains the cleavage site for ADAMTS13 (see Tsai, R. and Interlandi, G. “Oxidation shuts down an auto-inhibitory mechanism of von Willebrand factor” Proteins: Structure, Function, and Bioinformatics 89 (6): 731-741, June 2021).

FIG. 2 is a schematic representation of a VWF monomer domains: D′, D3, A1, A2, and A3.

FIG. 3 is a schematic illustration of lumacaftor bridging domains A1 and A2.

FIG. 4 is a schematic illustration showing that contacts between side chains and aromatic rings stabilize the interaction between lumacaftor and the A1 and A2 domains.

FIG. 5 is a schematic illustration of an ELISA for testing the ability of compounds to affect the VWF binding activity.

FIG. 6 shows the results of titration with oxidation performed in-plate. The results show the increase in VWF binding activity with increasing concentration of HOCl in ELISA. The binding experiments were performed with a 2×GpIbα concentration. A “+” indicates a difference that is marginally statistically significant (p-value between 0.05 and 0.1) while a “*” denotes a statistically significant difference (p-value≤0.05) when comparing oxidized to non-oxidized (no ox) VWF.

FIGS. 7A and 7B show the results of titration with oxidation performed in-vial and compare the effects of oxidation on VWF function and methionine oxidation. The results show increasing concentrations of HOCl corresponded to increasing binding activity between VWF and GpIbα (FIG. 7A). The binding experiments were performed with a 5×GpIbα concentration. A “+” indicates a difference that is marginally statistically significant (p-value between 0.05 and 0.1) while a “*” denotes a statistically significant difference (p-value≤0.05) when comparing oxidized to non-oxidized (no ox) VWF. “GpIbα only” indicates that no VWF was used. The concentration of oxidant also correlated with an increased ratio of oxidized methionine residues in the A1, A2 and A3 domains measured in mass spectrometry experiments (FIG. 7B). NanoLC-MS/MS of methionine residues as a function of HOCl concentration was performed. The values for M1385 are reported for variants containing alanine or threonine at position 1381 because VWF found in humans consists of a population with both variants. Vertical dashed lines separate residues located within A1, A2 and A3 domains.

FIG. 8 compares the effects of oxidation on VWF function in the absence and presence of lumacaftor. Lumacaftor reduced the binding of GpIbα to oxidized VWF while binding to unoxidized VWF was not affected.

FIG. 9 compares the effects of oxidation on VWF function: lumacaftor reduced the binding of GpIbα to oxidized VWF while binding to unoxidized VWF was not affected. Relative to the results shown in FIG. 8, the results shown were obtained using half the concentration of GpIbα as in FIG. 8 (38.5 nM instead of 77 nM).

### DETAILED DESCRIPTION

In one aspect, the present disclosure provides lumacaftor as a VWF ligand that prevents thrombosis while maintaining hemostasis. In the method, a therapeutically effective amount of lumacaftor or a pharmaceutically acceptable salt thereof is administered to a subject in need thereof.

As used herein, the term “lumacaftor” refers to 3-[6-[[1-(2,2-difluoro-1,3-benzodioxol-5-yl)cyclopropanecarbonyl]amino]-3-methylpyridin-2-yl]benzoic acid having the structure shown below:

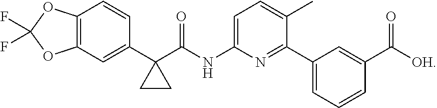 [SMILES: Cc1ccc(NC(=O)C2(c3ccc4c(c3)OC(F)(F)O4)CC2)nc1-c1cccc(C(=O)O)c1]

In the methods described herein, lumacaftor or a pharmaceutically acceptable salt thereof is administered to a subject. As used herein, the term “pharmaceutically acceptable salt” refers to an ion of lumacaftor (e.g., carboxylate or pyridinium) acceptable for pharmaceutical administration. Lumacaftor salts can be formed by the neutralization reaction of an acid and a base. For lumacaftor's carboxylic acid functionality, salts can be derived from a variety of organic and inorganic counter ions well known in the art and include sodium, potassium, calcium, magnesium, ammonium, and tetraalkylammonium; and for lumacaftor's basic functionality, salts of organic or inorganic acids, such as hydrochloride, hydrobromide, tartrate, mesylate, acetate, maleate, and oxalate.

In another aspect, the disclosure provides a method for restoring the autoinhibitory function of von Willebrand factor (VWF) under pro-thrombotic inflammatory conditions. In the method, lumacaftor or a pharmaceutically acceptable salt thereof binds to a methionine sulfoxide at the A1-A2 inter-domain interface of VWF. The method comprises contacting VWF with an amount of lumacaftor or a pharmaceutically acceptable salt thereof effective to bind to a methionine sulfoxide at the A1-A2 inter-domain interface of VWF. In certain embodiments, the methionine sulfoxide to which lumacaftor or a pharmaceutically acceptable thereof binds is the oxidized form of M1495.

The methods described herein are selective. Contacting VWF with an effective amount of lumacaftor or a pharmaceutically acceptable salt thereof does not result in interfering with and does result in maintaining the hemostatic function of unoxidized VWF. In certain embodiments, this selectivity is a result of lumacaftor or a pharmaceutically acceptable salt thereof binding to unoxidized M1495 significantly less strongly than to oxidized M1495.

In a further aspect, the present disclosure provides a method based on an enzyme-linked immunosorbent assay (ELISA) to measure the binding activity of VWF to GpIbα comparing oxidized and unoxidized VWF in the absence and the presence of lumacaftor. Mass spectrometry was used to ascertain whether the increased VWF activity with oxidation was correlated to the rate of methionine oxidation. The advantage of ELISA is that it allows comparing multiple conditions at the same time as each plate used contains 96 wells, and more conditions can be compared with plates containing an even larger number of wells. Hence, the method described herein serves as the basis for a high-throughput screening assay.

The disclosure provides a method for measuring the binding activity of VWF to GpIbα comparing oxidized and unoxidized VWF in the absence and the presence of a therapeutic candidate. The method is a solid-phase assay that relies on measuring the amount of GpIbα bound to VWF immobilized on a solid support as a function of therapeutic candidate. In one embodiment, the method comprises measuring the amount of GpIbα bound to VWF immobilized on a solid support. In the method, binding of GpIbα to oxidized VWF is compared to binding of GpIbα to unoxidized VWF, each in the absence or presence of a therapeutic candidate. A successful therapeutic drug candidate inhibits the binding of GpIbα to oxidized VWF while the binding strength of GpIbα to unoxidized VWF is left unaltered, i.e., the therapeutic drug candidate is selective to the oxidation state of VWF; the greater the inhibition of the binding of GpIbα to oxidized VWF while the binding strength to unoxidized VWF is unaltered (i.e., selectivity), the more potent the therapeutic candidate. In the assay, oxidized VWF is immobilized and then treated with an oxidizing agent, such as HOCI, or left unoxidized. In the assay, GpIbα bound to VWF immobilized on a solid support is determined by measuring the signal from a reporting agent that binds to the bound GpIbα. In certain embodiments, the reporting agent is an enzyme-labeled antibody that binds to the polyhistidine tag (His-tag) of the bound GpIbα, and the signal generated by the reporting agent is an enzyme product measured by absorbance. Representative reporting agents include horse radish peroxidase (HRP) conjugates of His-tag antibodies.

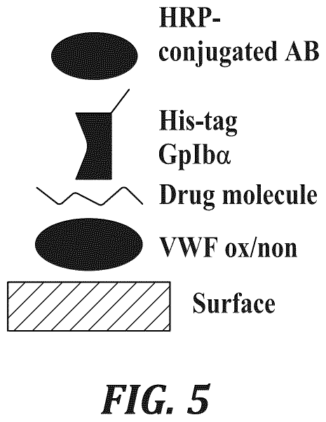

A schematic illustration of the ELISA is shown in FIG. 5 and described in the Methods section below. Briefly, VWF is adsorbed on the surface of a 96-well plate, the plate in incubated overnight with 1% BSA, the therapeutic candidate to be tested (e.g., lumacaftor) is added to test wells, recombinant His-tagged GpIbα is added, and binding of GpIbα to VWF is detected through a HRP-conjugated His-tag antibody (i.e., measuring the absorbance of the HRP product). In the assay, VWF is treated with various concentrations of HOCl (e.g., 50-100 μM) or left unoxidized. Oxidation is quenched with free methionine.

As described herein, lumacaftor is demonstrated to be a ligand for selective targeting of von Willebrand factor (VWF) under oxidizing (inflammatory) conditions. VWF is key in the initial stage of blood coagulation, in particular at high shear, where VWF tethers platelets and slows them down and integrins bring platelets to a final stop. Although mutations that deactivate VWF lead to von Willebrand Disease, a minor bleeding disorder, VWF hyperactivation leads to pathological thrombus formation, which can be due to disruption of the down-regulation mechanism or oxidizing conditions.

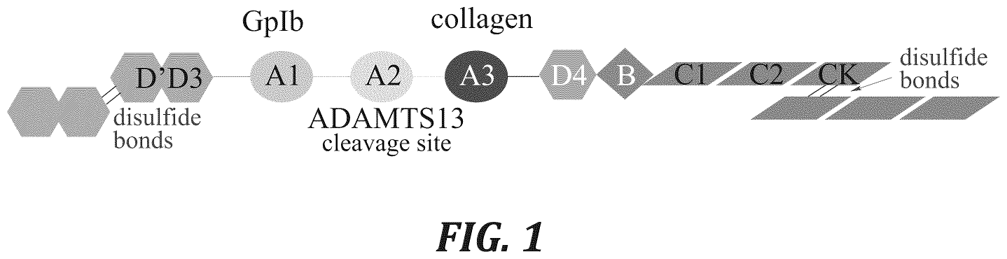

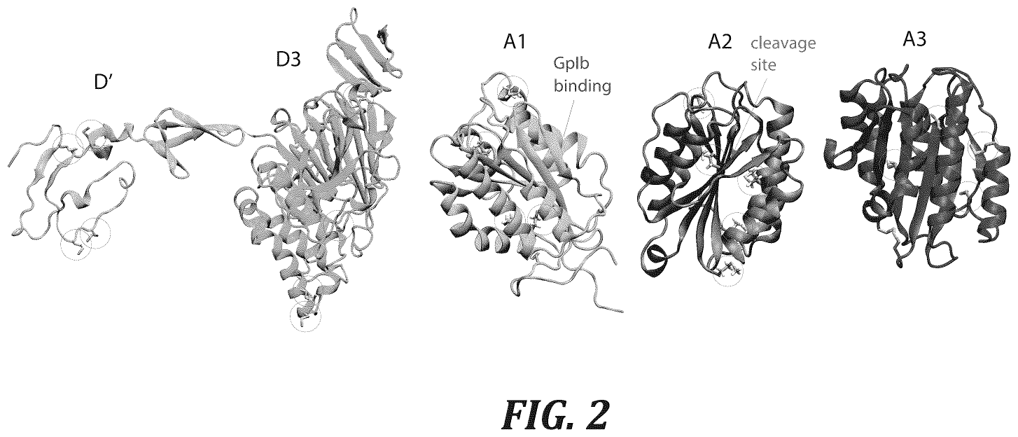

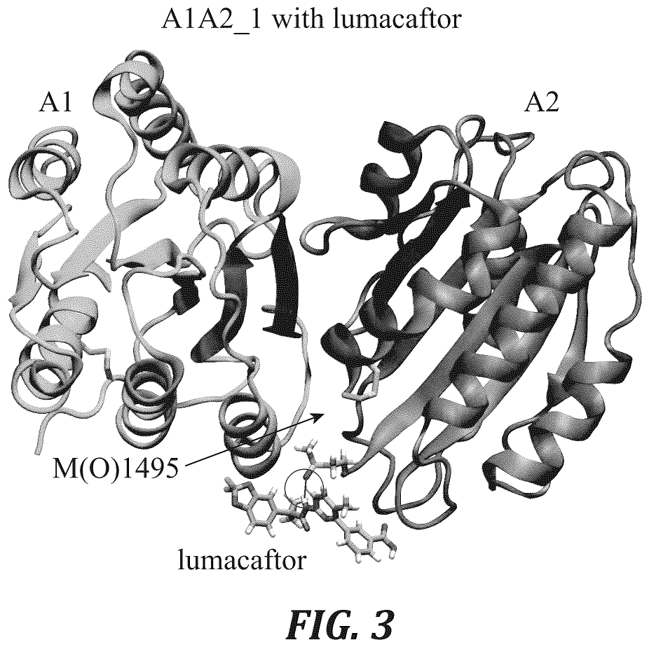

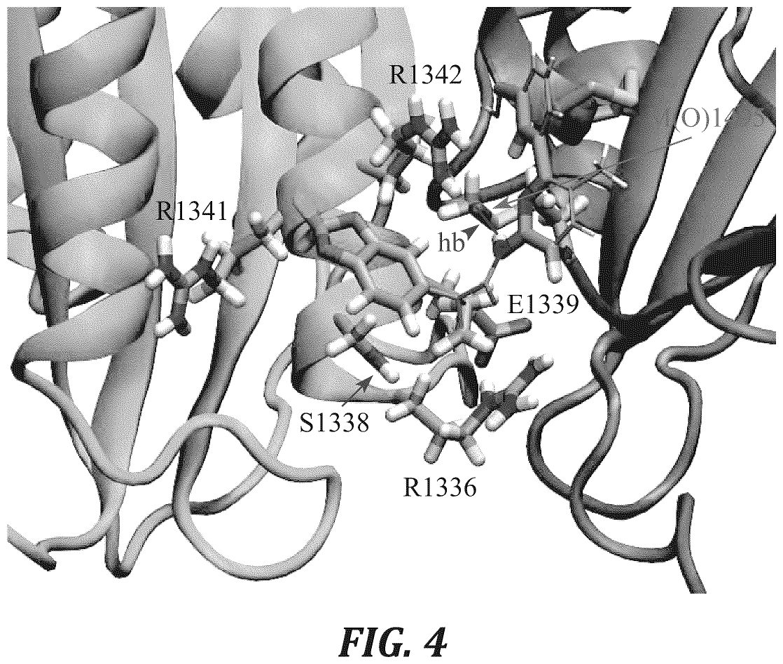

A schematic illustration of VWF structure is shown in FIG. 1: a VWF monomer is shown indicating substrates of the A1 (GpIbα) and A3 (collagen) domains and illustrating that the A2 domain contains the cleavage site for ADAMTS13. The VWF domains D′, D3, A1, A2, and A3 are illustrated in FIG. 2. The bridging of VWF domains A1 and A2 by lumacaftor is illustrated in FIG. 3. FIG. 4 illustrates contacts between side chains and aromatic rings stabilizing the interaction between lumacaftor and domains A1 and A2.

Inflammation and oxidation affect VWF function. Inflammation activates inflammatory cells like neutrophils, which produce hydrogen peroxide. Myeloperoxidase converts hydrogen peroxide to hypochlorus acid, an oxidant. Hypochlorus acid oxidizes methionine residues in plasma proteins (methionine to methionine sulfoxide). Oxidation is known to lead to a pro-thrombotic state. Regarding VWF, oxidation reduces ADAMTS13 cleavage ability of the A2 domain of VWF and oxidation of full-length VWF increases its platelet agglutination ability.

Experimental evidence shows that the A1 domain of VWF is inhibited by neighboring domains, that methionine residues are present either in the core or on the surface of domains, and neighboring A2 domain inhibits A1 binding to GpIbα, but oxidation removes this function.

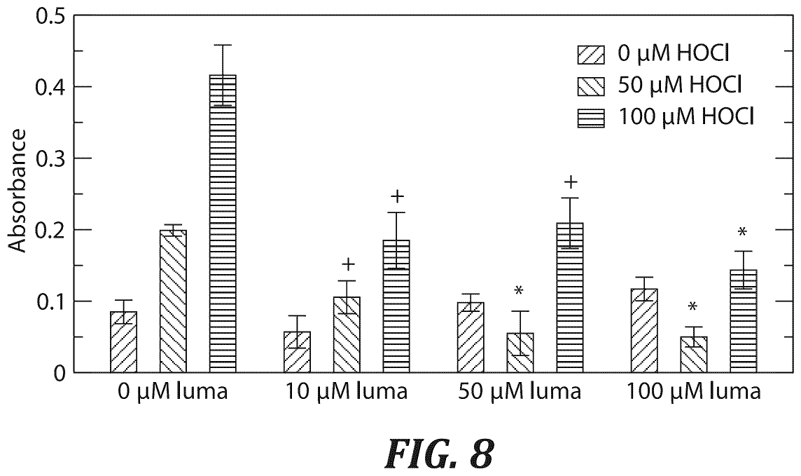

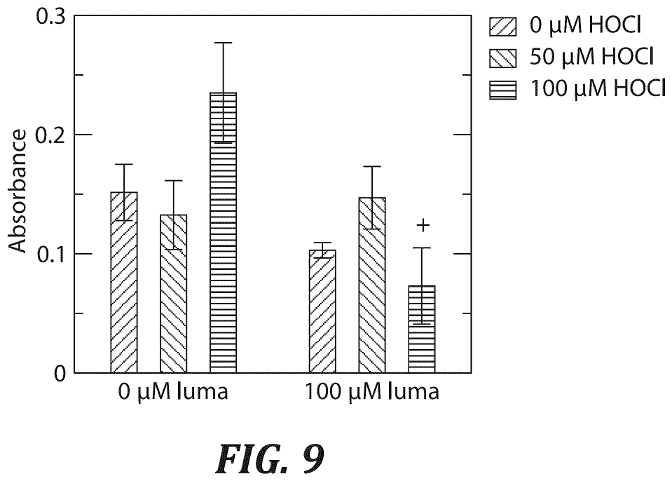

The present disclosure addresses the disadvantage of unwanted bleeding associated with current anti-thrombotic therapeutics and provides a molecule, lumacaftor, that binds to the oxidized A1A2 domains and restores the inhibitory function that the A2 domain normally has on the A1 domain (FIGS. 8 and 9). As described herein, lumacaftor binds significantly stronger to A1A2 when M1495 is oxidized and therefore compensates for the destabilizing effect of methionine oxidation in VWF.

The present disclosure demonstrates the use of lumacaftor as an anti-thrombotic agent that prevents thrombosis while maintaining hemostasis. As such, lumacaftor may be an alternative to Eliquis® (apixaban) with reduced or absent side effect of hemorrhage, such as intracranial bleeding. Lumacaftor may also be an alternative to caplacizumab used to treat thrombotic thrombocytopenia purpura (TTP), which is due to hyperactivity of VWF (ADAMTS13 deficiency).

Activation of VWF Correlates with Methionine Oxidation

In order to design drugs that target VWF under oxidizing conditions, it is necessary to evaluate whether the increased activity of VWF in the presence of HOCl is linked to the increased rate of methionine oxidation. Establishing such a link makes it possible to use structure-based drug design to narrow down from a large database a set of molecules to be tested in vitro as previously described (Interlandi, G. “Exploring ligands that target von Willebrand factor selectively under oxidizing conditions through docking and molecular dynamics simulations” Proteins 92 (11): 1261-1275 November 2024). Increased platelet-binding activity of VWF due to oxidation through HOCl has been previously reported using a ristocetin-induced platelet agglutination (RIPA) assay. However, ELISA is a much more convenient assay to test the activity of VWF under various conditions because it allows measuring multiple combinations of oxidant and drug concentrations in one single plate. Accordingly, the following was tested: (1) whether the oxidation-induced activation of VWF observed in RIPA can be recapitulated in an ELISA, and (2) whether the increased activation is related to the oxidation rate of methionine residues.

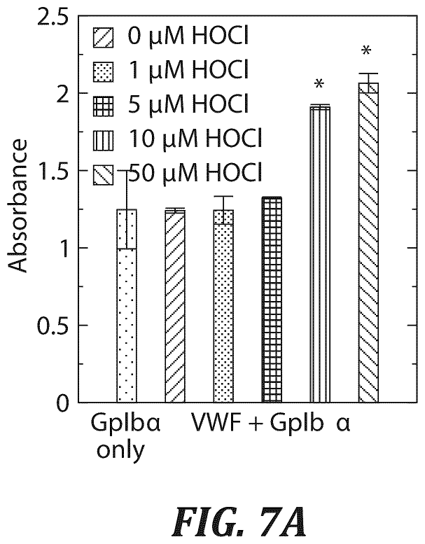

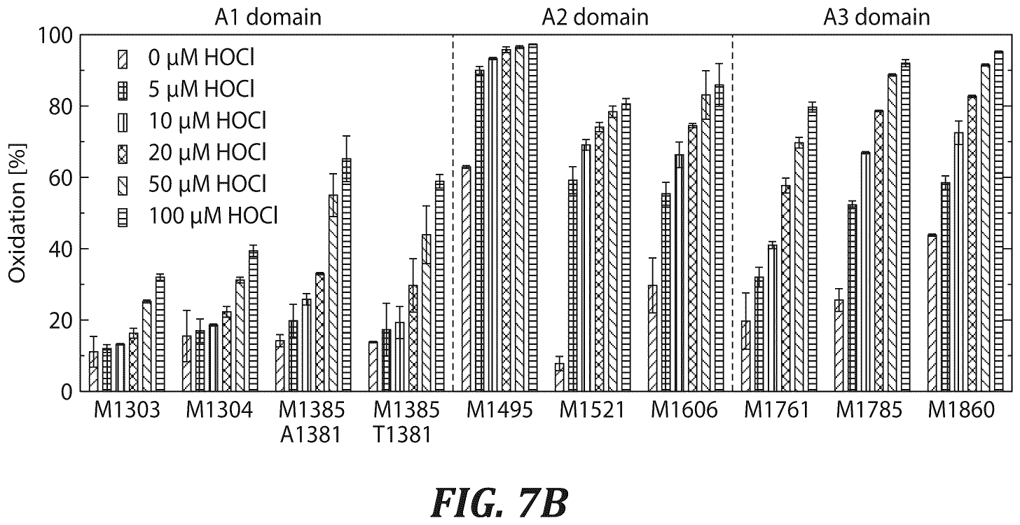

In order to facilitate nanoLC-MS/MS analysis of the methionine oxidation rate in VWF, oxidation was performed in-vial, as described below. It was observed that increasing concentrations of HOCl increased the activity of VWF in binding GpIbα (FIG. 7A). Furthermore, increasing concentrations of HOCl also caused increasing rates of oxidation of specific methionines for which it was possible to determine the oxidation state in our nanoLC-MS/MS assay (FIG. 7B). Hence, increasing concentrations of HOCl cause both an increase in binding activity and a higher rate of methionine oxidation.

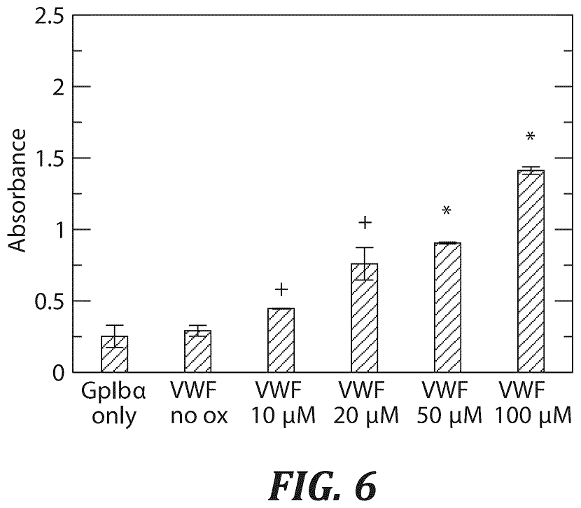

In order to test whether oxidation increases the activity of VWF also when HOCl is added after VWF is adsorbed to the plate, an ELISA was performed where various concentrations of HOCl were added in-plate after incubating overnight with 1% BSA-PBS. We observed that VWF binding to GpbIα was observed to increase with increasing oxidant concentration where 50 μM and 100 μM HOCl produced results that were significantly different from the activity of non-oxidized VWF (FIG. 6). The following ELISA experiments were performed with in-plate oxidation.

### ELISA Tests of Lumacaftor

After coating wells with VWF and incubating overnight with blocking buffer, different concentrations of lumacaftor were added to the plate after it was either left unoxidized or oxidized with various amounts of HOCI. While oxidation increased the binding activity of VWF, lumacaftor decreased it significantly. Oxidation was observed to also increase the background non-specific adhesion of GpIbα to the plate although with a significantly smaller effect than in the presence of adsorbed VWF. In fact, subtracting the background for each respective HOCl concentration still yielded statistically significant differences when comparing oxidized to unoxidized VWF and the effect of lumacaftor when added to oxidized VWF (FIGS. 8 and 9). Furthermore, it was possible to detect binding of GpIbα even to unoxidized VWF (FIGS. 8 and 9). This observation indicates that it is possible to measure the activity of unoxidized VWF with this assay, which allows testing the effect of a drug also on unoxidized VWF, which is normally less active. Notably, lumacaftor did not alter the function of unoxidized VWF (FIGS. 8 and 9) making it a suitable candidate for a therapeutic drug that prevents thrombosis while maintaining hemostasis.

As noted above, it is desirable to discover a therapeutic that prevents pathological thrombus formation while allowing the hemostatic response in case of acute injury. Due to its central role in initiating the blood clotting process, the protein VWF serves as a key target for anti-thrombotic interventions. Oxidizing agents released during a pro-thrombotic inflammatory state have been shown to increase the platelet-binding function of VWF and to convert methionine residues to methionine sulfoxide. Hence, the question arises whether it is possible to find a drug that is sensitive to the oxidation state of VWF, inhibiting it only under the conditions that cause a pro-thrombotic environment.

Previous studies have suggested that oxidation of methionine residues removes the inhibitory function that the A2 domain normally has on the A1 domain. In particular, methionine residues located at the A1-A2 domains interface are converted to methionine sulfoxide in the presence of oxidizing agents disrupting the interaction between A1 and A2 domains. It is therefore plausible that designing a drug that binds to oxidized methionine residues at the A1-A2 interface may act as a glue between the two domains and restore the inhibitory function of the A2 domain on the A1 domain.

The ELISA described herein was developed to test the effect of lumacaftor on the function of non-oxidized and oxidized VWF. MS-LC analysis revealed that the increase in VWF activity due to the addition of HOCl in the ELISA correlated with an increase in the rate of methionine oxidation (FIGS. 7A and 7B) indicating that such assay can be used to test the activity of VWF under oxidizing conditions. While computational high-throughput screening can be helpful to pre-select possible candidates from a very large database of molecules that have a particular effect on a protein, an efficient in vitro assay is required to rapidly screen the computationally pre-selected drugs on the function of a protein under various conditions such as in this case non-oxidized and oxidized VWF. The ELISA-based method described herein allows simultaneously testing multiple drugs under various concentrations comparing non-oxidized and oxidized VWF, and for that reason can serve as a high-throughput in vitro screening assay for drugs. The method was employed to test the effect of lumacaftor on VWF. Analysis of the results indicated that lumacaftor reduced the function of oxidized VWF while leaving VWF function unaltered under normal conditions (FIGS. 8 and 9).

Three conclusions emerge from the study. The first conclusion is that it is possible to design an ELISA to test the binding activity of VWF to the platelet surface receptor GpIbα. Previous studies have used a dynamic flow assay to measure the activity of full-length VWF or recombinant VWF constructs, which more closely mimics the effects of tensile force due to flowing blood. However, as shown previously and described herein (FIGS. 7A and 7B), oxidation is also capable of activating VWF so that its GpIbα binding properties can be measured in a static assay. Furthermore, the ELISA was also able to detect binding of GpIbα to VWF not treated with HOCl (FIGS. 8 and 9) making it possible to test whether a drug alters VWF function under both oxidizing and non-oxidizing conditions. Such an ELISA-based approach allows for a rapid screening of different drugs under different conditions because such a static assay is simpler, less time intensive and less costly to setup then a dynamic flow assay as used in previous studies.

The second conclusion, which is derived from the first, is that VWF can be activated also through adsorption on a polystyrene surface as observed in the ELISA assay (FIGS. 8 and 9) in the absence of any modulator such as tensile force, oxidation or the drug ristocetin, which is known to be a potent activator of VWF. In a previous study that investigated how different material surfaces affect the GpIbα-binding function of the VWF A1 domain, polystyrene and tissue-culture polystyrene were found to increase the activity of A1 compared to glass. However, there are no studies reported to date how different material surfaces affect the function of full-length VWF. In this study, it was observed that polystyrene can activate VWF.

The third conclusion is that lumacaftor reduces the binding activity of VWF when oxidized (FIGS. 8 and 9). Furthermore, the function of unoxidized VWF was unaltered by the addition of lumacaftor (FIGS. 8 and 9) highlighting that this drug selectively targets VWF under oxidizing conditions (i.e., lumacaftor is a potent inhibitor of VWF that works selectively in the presence of inflammation-induced oxidizing agents).

### Materials and Methods

### Reagents for ELISA

Purified full-length human von Willebrand factor was purchased from Prolytix (HCVWF-0190). Recombinant human GpIbα containing a histidine tag at the C-terminus was purchased from bio-techne (4067-GP). Generally, stock solutions were prepared using 0.2% BSA-PBS as the buffer. The ELISA was performed using Immulon 4 HBX 96-well plates.

### ELISA Experiments

Wells of a 96-well polystyrene plate were coated each with 100 μL of a solution containing 1 μg of VWF. Subsequently, the plate was incubated at 37° C. for 90 minutes, after which the coated wells were washed three times with PBS as after each incubation step. Assuming a molecular weight of a VWF monomer to be approximately 260 kDa, the VWF solution used here corresponds to a concentration of about 38.5 nM of VWF monomers. It is important to note that VWF is multimeric, but each monomer contains one A1 domain, i.e., one binding site for GpIbα. The protein VWF was either left unoxidized or was oxidized with HOCI. Oxidation of VWF was either performed in-vial before adding the solution to the wells, or in-plate after VWF had been adsorbed on the plate. To perform in-vial oxidation, HOCl was added to the VWF solution to achieve the final concentration indicated in the experiment and the vial was rotated at 37° C. for 60 minutes, followed by quenching with excess free methionine rotating at 37° C. for 15 minutes. In-plate oxidation was performed by adding 100 μL of HOCl at the concentration indicated in the experiment and incubating for 60 minutes at 37° C. This was followed by quenching with excess free methionine while incubating for 15 minutes. To block the wells, 1% BSA-PBS was added to the wells and the plate was incubated overnight at 4° C.

To test the effect of drug molecules, a 100 μL solution containing a drug at the indicated concentration was added to specific wells of the plate and incubated at 37° C. for 60 minutes. Finally, recombinant GpIbα was added to all wells at a concentration of either 1-fold, two-fold or five-fold the concentration of the VWF solution used in the first incubation step (38.5 nM). The different concentrations of GpIbα were labeled as “1×GpIbα”, “2×GpIbα”, and “5×GpIbα”, respectively. Then, a horseradish peroxidase-conjugated anti-histidine antibody (BioLegend Clone J099B12) was added to the wells and incubated at 37′C for 60 minutes. Subsequently, TMB was added, the reaction was stopped with a stop reagent (Sigma-Aldrich S5814) and the plate was read at 450 nm.

### Nano Flow Liquid Chromatography Tandem Mass Spectrometry

Human VWF oxidized samples were reduced with dithiothreitol (Bio-RAD), alkylated with iodoacetamide (Bio-RAD), and digested with Trypsin/LysC (Promega). The resulting tryptic peptides were desalted using a C18 cartridge (3 M Science) and analyzed by nano flow liquid chromatography tandem mass spectrometry (nanoLC-MS/MS) using a Thermo Scientific Orbitrap Fusion™ Lumos™ Tribrid mass spectrometer coupled with a Waters nanoACQUITY Ultra Performance LC system. Peptides were separated at a flow rate of 300 nL/min on an ACQUITY UPLC M-Class HSS T3 Column (100×0.075 mm, 1.8 μm, Waters), using solvent A (0.1% formic acid in water) and solvent B (0.1% formic acid in acetonitrile). Peptides were eluted with a linear gradient: 5%-25% solvent B over 38 min, 25%-90% solvent B over 5 min, and 90%-95% solvent B over 15 min. Data acquisition was performed in positive ion mode with a Parallel Reaction Monitoring (PRM) method targeting predefined peptides. Peptide peak areas were quantified using Thermo Scientific™ Xcalibur™ software (v2.2). The percent oxidation of individual methionine residues was calculated by dividing the peak area of the methionine-containing oxidized peptide by the sum of the peak areas of both oxidized and unoxidized peptides.

As used herein, the term “about” refers to +5% of the specified value.

While illustrative embodiments have been illustrated and described, it will be appreciated that various changes can be made therein without departing from the spirit and scope of the invention.

1. A method for treating thrombosis in a subject, comprising administering to a subject in need thereof a therapeutically effective amount of lumacaftor or a pharmaceutically acceptable salt thereof.

2. A method for restoring the autoinhibitory function of von Willebrand factor (VWF) under pro-thrombotic inflammatory conditions, comprising binding lumacaftor or a pharmaceutically acceptable salt thereof to a methionine sulfoxide at the A1-A2 inter-domain interface of VWF.

3. The method of claim 2, wherein binding lumacaftor or a pharmaceutically acceptable salt thereof to the methionine sulfoxide comprises contacting VWF with an effective amount of lumacaftor or a pharmaceutically acceptable salt thereof.

4. The method of claim 2, wherein the methionine sulfoxide is the oxidized form of M1495.

5. The method of claim 2, wherein contacting VWF with an effective amount of lumacaftor or a pharmaceutically acceptable salt thereof does not interfere with and does maintain the hemostatic function of unoxidized VWF.
